(19) United States
(12) Patent Application Publication
(10) Pub. No.: US 2010/0217288 A1
(43) Pub. Date: Aug. 26, 2010

(54) Transesophageal gastric reduction method and device for practicing same

(76) Inventors: Steve G. Baker, Redmond, WA (US); Raymond Michael Wolniewicz, III, Redmond, WA (US); Sean Totten, Kirkland, WA (US); Clifton A. Alferness, Port Orchard, WA (US)

Correspondence Address: HOEKENDIJK & LYNCH, LLP, P.O. BOX 4787, BURLINGAME, CA, 94011-4787, US

(21) Appl. No.: 12/799,571

(22) Filed: Apr. 27, 2010

Related U.S. Application Data
(63) Continuation of application No. 11/352,431, filed on Feb. 10, 2006.

(51) Int. Cl. A61B 7/00

(52) U.S. Cl. 606/151

(57) ABSTRACT

A gastric reduction pouch of a stomach is formed by gathering stomach tissue circumferentially from within the stomach to form a reduced diameter stomach section. A plurality of fasteners are deployed within the gathered stomach tissue to maintain the reduced diameter stomach portion. The gathering step may include folding the stomach tissue to produce a plurality of stomach tissue folds.

### FIELD OF THE INVENTION

The present invention is generally directed to a therapy for treating obesity. The present invention is more particularly directed to a transesophageal gastric reduction method and device for performing gastric reduction surgery while minimizing surgical invasion.

### BACKGROUND OF THE INVENTION

Obesity is a complex chronic disease involving environment, genetic, physiologic, metabolic, behavioral and psychological components. It is the second leading cause of preventable death in the United States.

Obesity affects nearly one-third of the adult American population (approximately 60 million). The number of overweight and obese Americans has continued to increase since 1960. The trend is not slowing down. Today, 64.5% of adult Americans are categorized as being overweight or obese. Each year, obesity causes at least 300,000 excess deaths in the United States, and healthcare costs of American adults with obesity amounted to approximately $100,000,000,000 (100 billion dollars).

Obesity is not limited to the United States but is increasing worldwide. It is increasing worldwide in both developing and developed countries and is thought to be caused by environmental and behavioral changes resulting from economic development, modernization, and urbanization. Obesity is increasing in children as well. It is believed that the true health consequences of obesity have not yet become totally apparent.

Obesity is currently treated by dietary therapy, physical activity, behavioral therapy, drug therapy, and combinations thereof. Dietary therapy involves instruction on how to adjust a diet to reduce the number of calories eaten. Physical activity strategies include use of aerobic exercise, brisk walking, jogging, cycling, and swimming. Behavioral therapy involves changing diet and physical activity patterns and habits to new behaviors that promote weight loss. Drug therapy is most often used only in conjunction with appropriate lifestyle modifications.

One last treatment for obesity is surgery. Surgery is a treatment option, which is generally reserved for persons with severe obesity and those who are morbidly obese. In addition, surgery is not generally performed until other methods of weight loss have been attempted and have been found to be ineffective. Persons who are severely obese are generally unable to physically perform routine daily activities, whether work-related or family functions and have a severely impaired quality of life due to the severity of their obesity.

Most obesity surgeries involve making changes to the stomach and/or small intestines. Currently, there are two types of obesity surgery: (1) restrictive; and (2) combined restrictive and malabsorptive. Operative procedures have been developed for each type of surgery. Each type of surgery has its own risks and side effects.

In restrictive surgery, bands or staples are used to create food intake restriction. The bands or staples are surgically placed near the top of the stomach to section off a portion that is often called a stomach pouch. A small outlet, about the size of a pencil eraser, is left at the bottom of the stomach pouch. Since the outlet is small, food stays in the pouch longer and the feeling of fullness lasts for a longer time. Current operative procedures for restrictive surgery include vertical banded gastroplasty, gastric banding, and laparoscopic adjustable gastric banding. In vertical banded gastroplasty, a stomach pouch is surgically created. In gastric banding, a band is used to create the stomach pouch. In laparoscopic gastric banding, a less invasive procedure, smaller incisions are made to apply the band. The band is inflatable and may be adjusted over time.

Each of the foregoing therapies for severe obesity has its risks and side effects. Each is invasive surgery and hence exhibits the risks commonly associated with all surgical procedures. Complications may include leaking of stomach juices into the abdomen, injury to the spleen, band slippage, erosion of the band, breakdown of the staple line, and stomach pouch stretching from overeating.

However, reductive surgery has proven successful. About 80% of patients lose some weight and 30% reach a normal weight. Hence, the benefits of gastric reduction surgery are generally believed to outweigh the attendant risks and potential complications.

The present invention is directed to an alternative method and device for achieving gastric reduction. As will be seen hereinafter, the method does not require surgical incisions and is thus less invasive than previous reduction therapies.

### SUMMARY OF THE INVENTION

The invention provides a method of forming a gastric reduction pouch of a stomach comprising the steps of gathering stomach tissue circumferentially from within the stomach to form a reduced diameter stomach section and deploying a plurality of fasteners within the gathered stomach tissue to maintain the reduced diameter stomach portion. The gathering step may include folding the stomach tissue to produce a plurality of stomach tissue folds.

The folding step may include forming a plurality of stomach tissue folds that are substantially parallel to each other. The stomach tissue folds are preferably substantially axially disposed.

The fastening step may include fastening at least two adjacent folds with a common fastener. The fastening step may also include deploying at least two fasteners in one of the folds.

The stomach tissue folds may alternatively be non-axially disposed. At least two adjacent stomach tissue folds may be overlapped. Also, the fastening step may include deploying at least two fasteners in one of the folds.

The invention further provides a method of forming a gastric reduction pouch of a stomach comprising the steps of forming a plurality of pleats in stomach tissue circumferentially from within the stomach to form a reduced diameter stomach section, and deploying at least one fastener within each pleat of stomach tissue to maintain the reduced diameter stomach portion.

The invention still further provides a device for forming and maintaining tissue folds from within the stomach comprising an elongated member having a distal end for transoral placement in the stomach, a tissue gatherer carried on the distal end of the elongated member for placement into the stomach, the tissue gatherer defining a tissue chamber and including a tissue puller that pulls tissue into the tissue chamber to form a tissue fold within the tissue chamber, and a fastener deployer that directs a fastener into and through the tissue chamber for binding the tissue fold.

The tissue chamber may be dimensioned to form a tissue fold having facing major tissue surfaces extending coextensively from a fold line. The fastener deployer may then direct the fastener through the tissue chamber for being substantially transverse to the facing major tissue surfaces of the tissue fold. The tissue gatherer may be arranged for forming tissue folds that are substantially axial in orientation. However, the tissue folds may alternatively substantially non-axial in orientation.

The fastener deployer is preferably arranged to deploy a plurality of fasteners in the tissue fold.

The tissue chamber may include an elongated opening for receiving the stomach tissue to fold the stomach tissue. The tissue puller may include a vacuum gripper and the vacuum gripper may pull the stomach tissue through the elongated opening under vacuum to form the tissue folds. The vacuum may be pulled through the elongated member and the elongated member may include a valve at the elongated member distal end that seals under the vacuum. The elongated member preferably has a transverse dimension configured to permit an endoscope to pass there through and through the valve when the valve is open. The tissue puller may include a mechanical gripper and the mechanical gripper may pull the stomach tissue through the elongated opening to form the tissue folds. The mechanical gripper may comprise a helical coil.

The tissue chamber has a width defined by an outer wall opposite the elongated opening and length dimension, and the length dimension is greater than the width dimension. The elongated opening has a width dimension and the elongated opening width dimension and the tissue chamber width dimension are arranged to permit the stomach tissue to engage and seal against the outer wall under vacuum applied through the opening and on the stomach tissue.

The device may further comprise a tissue support within the tissue chamber that supports the stomach tissue fold during fastener deployment. The fastener deployer may include a guide lumen that guides a fastener deployment stylet through the stomach tissue fold. The tissue chamber may further include a stylet shield that receives the distal end of the fastener deployment stylet. The stylet shield may comprise a sheath. The device may further comprise a plurality of guide lumens for guiding a like plurality of fasteners through the stomach tissue fold.

The invention further provides a fastener assembly for use in a mammalian body comprising a first member, a second member, and a third member. The first, second and third members have first and second ends. The assembly further comprises a first connecting member fixed to each of the first and second members intermediate the first and second ends and extending between the first and second members and a second connecting member fixed to each of the second and third members intermediate the first and second ends and extending between the second and third members.

The first and second members are separated by the first connecting member and the second and third members are separated by the second connecting member. At least two of the first, second, and third members have a longitudinal axis and a through channel along the longitudinal axis arranged to be slidingly received on a tissue piercing deployment wire.

In another embodiment, a fastener assembly for use in a mammalian body comprises a first member, a second member, and a third member. The first, second and third members have first and second ends. A first connecting member, fixed to each of the first and second members intermediate the first and second ends, extends between the first and second members. A second connecting member fixed to each of the second and third members intermediate the first and second ends extends between the second and third members. Hence, the first and second members are separated by the first connecting member, and the second and third members are separated by the second connecting member. Each of the first, second and third members has a longitudinal axis and a through channel along the longitudinal axis arranged to be slidingly received on a tissue piercing deployment wire.

Each of the first, second and third members further includes a pointed tip. The connecting members are preferably flexible permitting the first, second and third members to be axially aligned when the first, second and third members are on the tissue piercing deployment wire.

The pointed tips may be conical and pointed in a common direction. The fastener assembly may be at least partially radio opaque.

The connecting members may be formed of elastic material. The first member, the second member, the third member and the connecting members may all be formed of plastic material. The first member, the second member, the third member and the connecting members are preferably all formed in one piece.

In another embodiment, a fastener assembly, for use in a mammalian body, comprises a first member, a second member, and a third member. The first, second and third members have first and second ends. A first connecting member fixed to each of the first and second members intermediate the first and second ends extends between the first and second members. A second connecting member fixed to each of the second and third members intermediate the first and second ends extends between the second and third members. The first and second members are separated by the first connecting member and the second and third members are separated by the second connecting member. Each of the first and third members has a longitudinal axis and a through channel along the longitudinal axis arranged to be slidingly received on a tissue piercing deployment wire.

Each of the first and third members further includes a pointed tip. The connecting members are flexible permitting the first and third members to be axially aligned when the first and third members are on the tissue piercing deployment wire. The pointed tips may conical and pointed in a common direction.

The fastener assembly may be at least partially radio opaque. The connecting members may be formed of elastic material. The first member, the second member, the third member and the connecting members may all be formed of plastic material. The first member, the second member, the third member and the connecting members may all be formed in one piece.

In another embodiment, a fastener for use in a mammalian body comprises a first member and a second member. The first and second members have first and second ends. A connecting member fixed to each of the first and second members intermediate the first and second ends extends between the first and second members. The first member has a longitudinal axis and a through channel along the axis arranged to be slidingly received on a tissue piercing deployment wire, and the second member has a plurality of segments arranged in a substantially common plane substantially transverse to the connecting member.

The first member further includes a pointed tip.

The connecting member is flexible permitting the second member to be next to the first member when the first member is on the tissue piercing deployment wire. The pointed tip may be conical. The pointed tip may comprise a sectioned portion.

The through channel may comprise a through bore. The segments of the second member may be divergent from the connecting member. The segments of the second member may be formed of resilient material for being initially disposed substantially parallel to each other prior to deployment and divergent after deployment.

The first member may further comprise a longitudinal slit communicating with the through channel. The first member, second member, and connecting member may all be formed of plastic material.

### BRIEF DESCRIPTION OF THE DRAWINGS

The features of the present invention which are believed to be novel are set forth with particularity in the appended claims. The invention, together with further features and advantages thereof, may best be understood by making reference to the following description taken in conjunction with the accompanying drawings, in the several figures of which like reference numerals identify identical elements, and wherein:

FIG. 1 is a front cross-sectional view of the esophageal-gastro-intestinal tract from a lower portion of the esophagus to the duodenum;

FIG. 2 is a perspective side view with portions cut away illustrating a reduced diameter stomach portion which may be formed according to the present invention for forming a gastric reduction pouch;

FIG. 3 is a cross sectional view of the reduced diameter stomach portion shown in FIG. 2;

FIG. 4 is another cross sectional view of the reduced diameter stomach portion shown in FIG. 2;

FIG. 5A is a cross sectional side view illustrating another reduced diameter stomach portion which may be formed according to the present invention for forming a gastric reduction pouch;

FIG. 5B is another cross sectional view of the reduced diameter stomach portion shown in FIG. 5A;

FIG. 6 is a cross sectional side view illustrating still another reduced diameter stomach portion which may be formed according to the present invention for forming a gastric reduction pouch;

FIG. 7 is a plan view of a device embodying the present invention;

FIG. 8 is a partial perspective view, with portions cut away, of the distal end of another device embodying the present invention;

FIG. 9 is a partial perspective view, with portions cut away, of the device of FIG. 8 in the process of folding stomach tissue in accordance with an embodiment of the present invention;

FIG. 10 is a partial perspective view, with portions cut away, of the device of FIG. 8 in the process of deploying a fastener through folded stomach tissue in accordance with an embodiment of the present invention;

FIG. 11 is a partial perspective view, with portions cut away, of the device of FIG. 8 after folding stomach tissue and deploying a fastener through the folded stomach tissue in accordance with an embodiment of the present invention;

FIG. 12 is a partial perspective view, with portions cut away, of the distal end of another device embodying the present invention;

FIG. 13 is a partial perspective view, with portions cut away, of the device of FIG. 12 after folding stomach tissue and deploying a pair of fasteners through the folded stomach tissue;

FIG. 14 is a partial perspective view, with portions cut away, of the distal end of another device embodying the present invention in the process of pulling stomach tissue to be folded towards the device;

FIG. 15 is a partial perspective view, with portions cut away, of the device of FIG. 14 after pulling the stomach tissue to be folded to the entrance of the device;

FIG. 16 is a partial perspective view, with portions cut away, of the device of FIG. 14 in the process of folding stomach tissue in accordance with an embodiment of the present invention;

FIG. 17 is a partial perspective view, with portions cut away, of the device of FIG. 14 in the process of deploying a fastener through folded stomach tissue in accordance with an embodiment of the present invention;

FIG. 18 is a partial perspective view, with portions cut away, of the device of FIG. 14 just after deploying a fastener through the folded stomach tissue;

FIG. 19 is a partial perspective view, with portions cut away, of the device of FIG. 14 after folding stomach tissue and deploying a fastener through the folded stomach tissue in accordance with an embodiment of the present invention;

FIG. 20 is a partial perspective view, with portions cut away, of the distal end of another device embodying the present invention in the process of folding stomach tissue in accordance with another embodiment of the present invention;

FIG. 21 is a partial perspective view, with portions cut away, of the device of FIG. 20 in the process of deploying a fastener through folded stomach tissue in accordance with an embodiment of the present invention;

FIG. 22 is a partial perspective view, with portions cut away, of the device of FIG. 20 just after deploying a fastener through the folded stomach tissue;

FIG. 23 is a partial perspective view, with portions cut away, of the device of FIG. 22 after folding stomach tissue and deploying a fastener through the folded stomach tissue in accordance with an embodiment of the present invention;

FIG. 24 is a perspective view, to an enlarged scale, of a fastener which may be employed according to an embodiment of the invention;

FIG. 25 is a side plan view, with portions cut away, of the fastener of FIG. 24 being delivered for deployment;

FIG. 26 is a perspective view of the fastener of FIG. 24 fully deployed;

FIG. 27 is a perspective view, to an enlarged scale, of another fastener which may be employed according to an embodiment of the invention;

FIG. 28 is a side plan view, with portions cut away, of the fastener of FIG. 27 being delivered for deployment;

FIG. 29 is a perspective view, to an enlarged scale, of another fastener which may be employed according to an embodiment of the invention;

FIG. 30 is a side plan view, with portions cut away, of the fastener of FIG. 29 being delivered for deployment;

FIG. 31 is a perspective view of the fastener of FIG. 29 fully deployed;

FIG. 32 is a perspective view, to an enlarged scale, of still another fastener which may be employed according to an embodiment of the invention;

FIG. 33 is a side plan view, with portions cut away, of the fastener of FIG. 32 being delivered for deployment;

FIG. 34 is a cross-sectional side view of the fastener of FIG. 32 fully deployed;

FIG. 35 is a partial perspective view, with portions cut away, of the distal end of another device embodying the present invention in the process of folding stomach tissue in accordance with another embodiment of the present invention;

FIG. 36 is a partial perspective view, with portions cut away, of the device of FIG. 35 in the process of deploying a fastener through folded stomach tissue in accordance with an embodiment of the present invention;

FIG. 37 is a partial perspective view, with portions cut away, of the device of FIG. 35 after folding stomach tissue and deploying a fastener through the folded stomach tissue in accordance with an embodiment of the present invention; and

FIG. 38 is a cross sectional side view illustrating another reduced diameter stomach portion, which may be formed by the device of FIG. 35 according to another embodiment of the present invention.

### DETAILED DESCRIPTION OF THE INVENTION

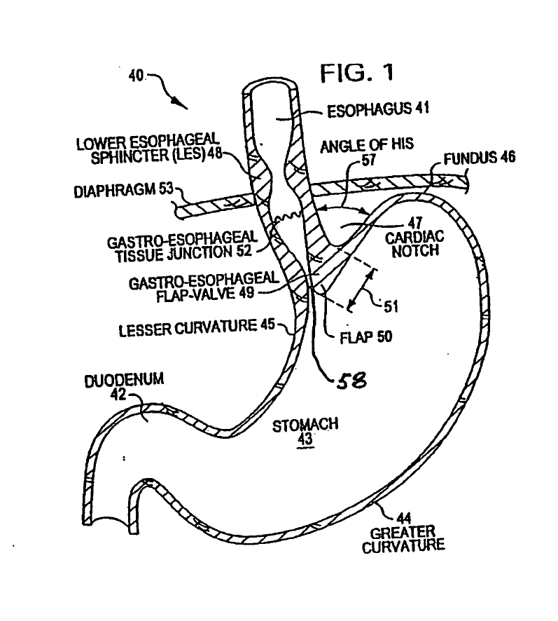

FIG. 1 is a front cross-sectional view of the esophageal-gastro-intestinal tract 40 from a lower portion of the esophagus 41 to the duodenum 42. The stomach 43 is characterized by the greater curvature 44 on the anatomical left side and the lesser curvature 45 on the anatomical right side. The tissue of the outer surfaces of those curvatures is referred to in the art as serosa tissue. As will be seen subsequently, the nature of the serosa tissue is used to advantage for its ability to bond to like serosa tissue. The fundus 46 of the greater curvature 44 forms the superior portion of the stomach 43, and traps gas and air bubbles for burping. The esophageal tract 41 enters the stomach 43 at an esophageal orifice 58 below the superior portion of the fundus 46, forming a cardiac notch 47 and an acute angle with respect to the fundus 46 known as the Angle of His 57. The lower esophageal sphincter (LES) 48 is a discriminating sphincter able to distinguish between burping gas, liquids, and solids, and works in conjunction with the fundus 46 to burp. The gastroesophageal flap valve (GEFV) 49 includes a moveable portion and an opposing more stationary portion. The moveable portion of the GEFV 49 is an approximately 180 degree, semicircular, gastroesophageal flap 50 (alternatively referred to as a “normal moveable flap” or “moveable flap”) formed of tissue at the intersection between the esophagus 41 and the stomach 43. The opposing more stationary portion of the GEFV 49 comprises a portion of the lesser curvature 45 of the stomach 43 adjacent to its junction with the esophagus 41. The gastroesophageal flap 50 of the GEFV 49 principally comprises tissue adjacent to the fundus 46 portion of the stomach 43, is about 4 to 5 cm long (51) at it longest portion, and the length may taper at its anterior and posterior ends. The gastroesophageal flap 50 is partially held against the lesser curvature 45 portion of the stomach 43 by the pressure differential between the stomach 43 and the thorax, and partially by the resiliency and the anatomical structure of the GEFV 49, thus providing the valving function. The GEFV 49 is similar to a flutter valve, with the gastroesophageal flap 50 being flexible and closeable against the other more stationary side.

The esophageal tract is controlled by an upper esophageal sphincter (UES) near the mouth for swallowing, and by the LES 48 and the GEFV 49 at the stomach. The normal anti-reflux barrier is primarily formed by the LES 48 and the GEFV 49 acting in concert to allow food and liquid to enter the stomach, and to considerably resist reflux of stomach contents into the esophagus 48 past the gastroesophageal tissue junction 52. Tissue aboral of the gastroesophageal tissue junction 52 is generally considered part of the stomach because the tissue protected from stomach acid by its own protective mechanisms. Tissue oral of the gastroesophageal junction 52 is generally considered part of the esophagus and it is not protected from injury by prolonged exposure to stomach acid. At the gastroesophageal junction 52, the juncture of the stomach and esophageal tissues form a zigzag line, which is sometimes referred to as the “Z-line.” For the purposes of these specifications, including the claims, “stomach” means the tissue aboral of the gastroesophageal junction 52.

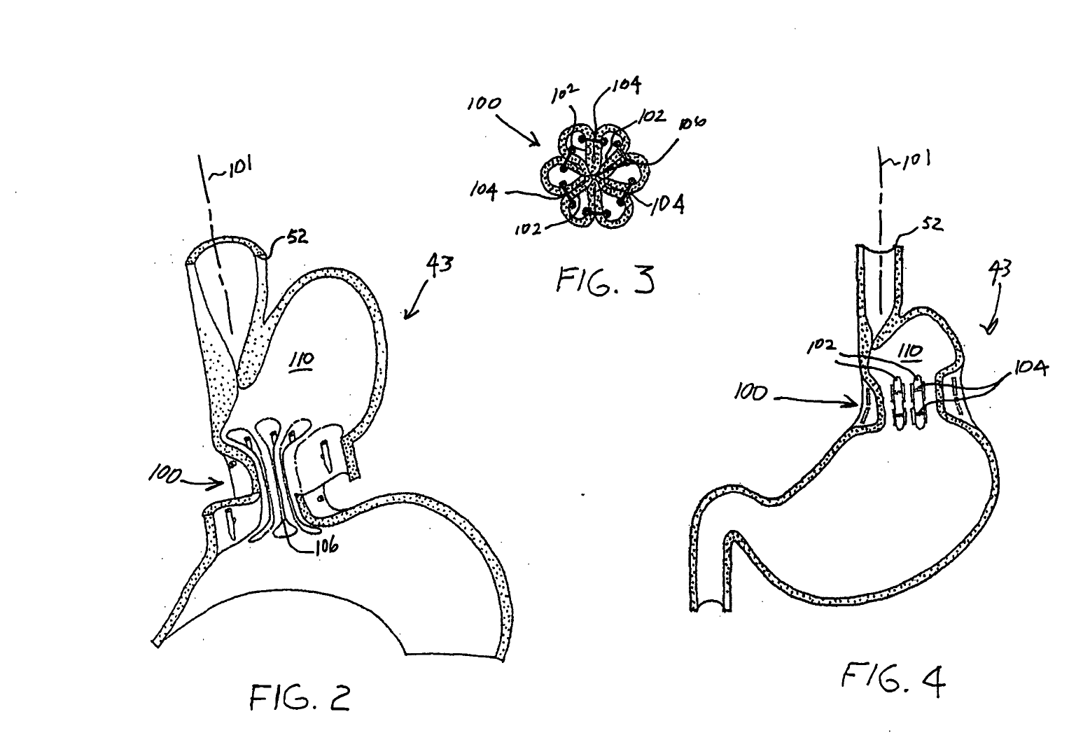

FIGS. 2-4 show a stomach with a reduced diameter portion 100 formed in accordance with an embodiment of the present invention to form a gastric reduction pouch 110. As may be noted in FIGS. 2 and 3, the gastric reduction pouch 110 is formed by a plurality of folds 102 made circumferentially about the stomach 43 aboral of the Z line 52. The folds 102 are formed by gathering the stomach tissue at circumferentially spaced points transorally from within the stomach. Devices according to various embodiments of the present invention for use in making the folds 102 and hence the reduced diameter portion 100 are described subsequently. Although the folds 102 are illustrated throughout the drawings as being equally spaced, it may be appreciated that the folds need not necessarily be equally spaced circumferentially without departing from the present invention.

The folds 102 are substantially parallel to each other and extend longitudinally in a substantially axial relation to the esophageal axis 101 in that their fold lines extend axially. This may be more clearly noted in FIG. 4. Each stomach tissue fold may be maintained by a pair of fasteners 104 as also shown in FIG. 4. Since the folds of stomach tissue are made inwardly, the outer surfaces of the stomach tissue come into contact when the folds are fixed by the deployment of one or more fasteners 104. This presents serosa tissue to serosa tissue to aid the fasteners with promoting tissue layer adhesion and the long term maintenance of the stomach tissue folds. The plurality of stomach tissue folds result in both a gastric reduction pouch 110 and an opening 106 communicating the interior of the gastric pouch with the rest of the gastric system.

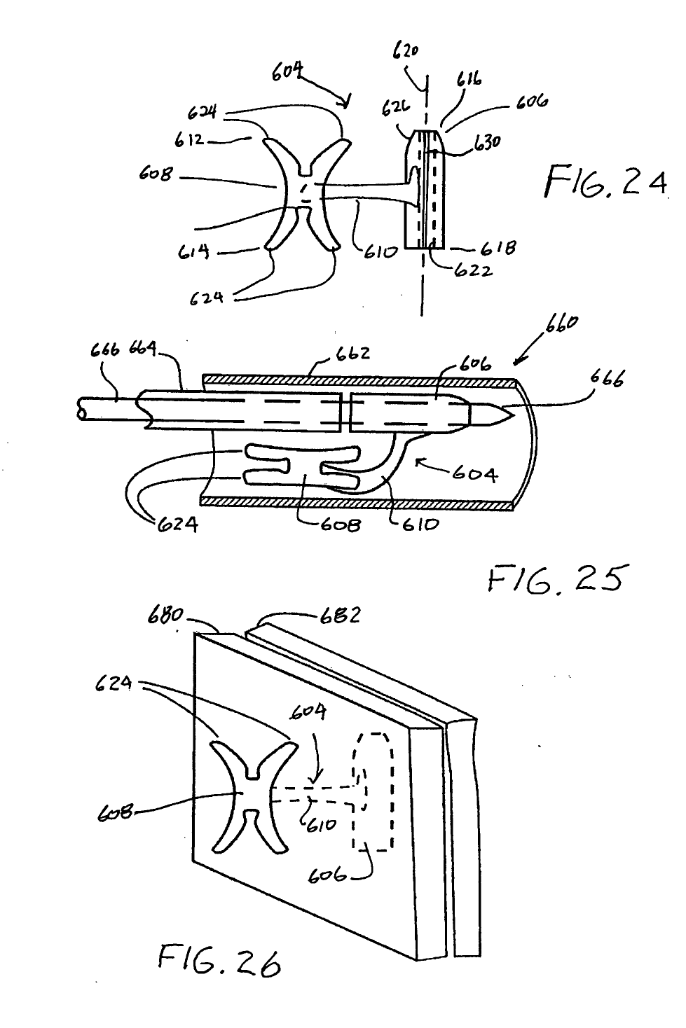

The fasteners may be of the type described in co-pending application Ser. No. 11/121,697, filed Jan. 25, 2005 titled SLITTED TISSUE FIXATION DEVICE AND ASSEMBLIES FOR DEPLOYING THE SAME which application is incorporated herein in its entirety. Other fasteners and fastener assemblies which embody further aspects of the invention and which may be used in securing the stomach tissue folds will be described herein with later reference to FIGS. 24-34.

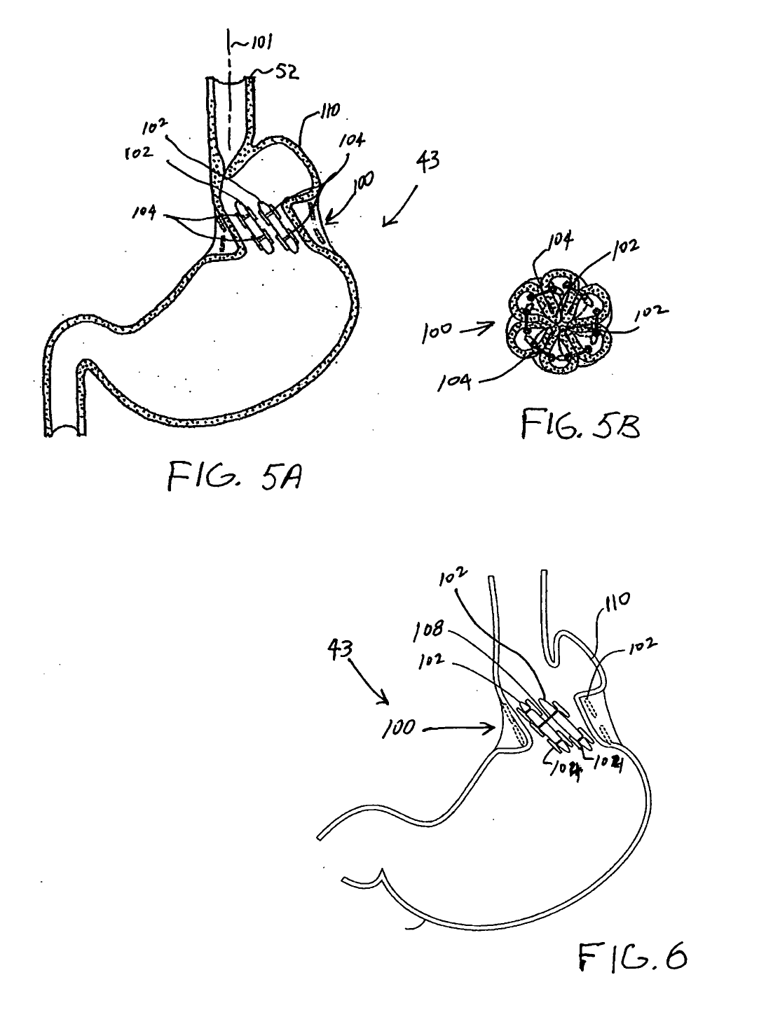

In FIGS. 5A and 5B it may be seen that the folds 102 that form the reduced diameter stomach portion 100 are still substantially parallel but here, however, they are non-axially disposed with respect to the esophageal axis 101. This causes the folds 102 to overlap in the axial direction. As in the previous embodiment, the folds are maintained by a pair of fasteners 104.

FIG. 6 provides a reduced diameter portion 100 similar to that of FIGS. 5A and 5B. Here, however, it may be noted that in addition to fasteners 102, at least one fastener, fastener 108, fastens two adjacent folds. This may serve to reduce the number of fasteners to be deployed to secure and maintain the reduced diameter stomach portion 100.

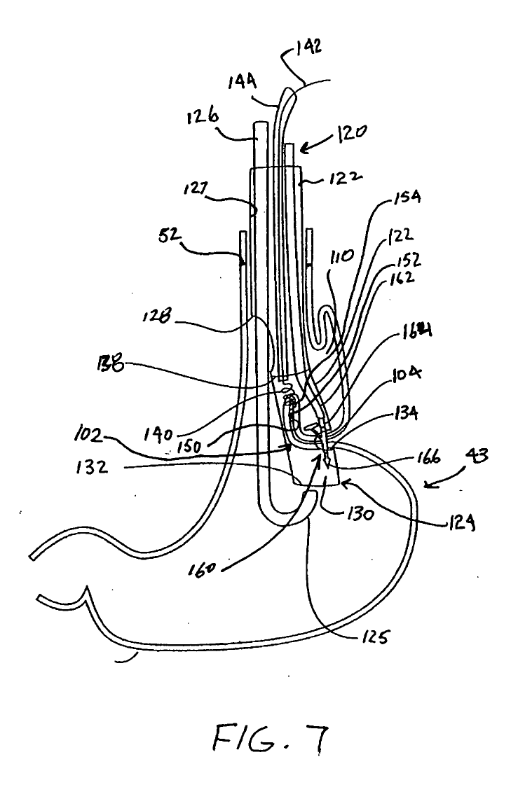

Referring now to FIG. 7, it shows a device 120 for forming and maintaining stomach tissue folds from within the stomach to form a gastric reduction pouch 110. The device 120 includes an elongated member 122 having a distal end 124 for transoral placement in the stomach 43. The elongated member includes a passageway 127 to permit an endoscope 126 to be fed down the device 120 and into the stomach. The endoscope has a reflexed portion 125 that enables visualization as necessary during the procedure to form the gastric reduction pouch 110. As may be noted in FIG. 7, the passageway 127 terminates in a port 128 that is intermediate the ends of the elongated member 122. Alternatively, the port 128 may be located at the distal end 124 of the elongated member 122. Preferably, the port 128 includes a seal that provides an effective seal when the endoscope is retracted from the device 120 or operatively positioned as shown.

The device 120 further includes a tissue receiving chamber 130 formed by the sidewalls of the elongated member 122, a distal seal 132, and a proximal seal 138. As may be noted in FIG. 7, the tissue receiving port 130 is elongated having a height or length dimension much greater than its width. The tissue receiving chamber 130 further includes a port 134 through which stomach tissue to be gathered and folded may enter the tissue receiving chamber 130. The port 134 is elongated and disposed substantially transverse to the elongated member center axis. This serves to form tissue folds that are substantially transverse to the longitudinal axis of the elongated member 122 and hence, the esophageal axis. Tissue pulled through the port 134 may be pulled axially upwardly in a proximal direction. Thus, together with the substantial length of the chamber 130 permit folds of substantial length to be developed.

As may further be noted in FIG. 7, the device is fed down into the stomach so that the port 134 is well distal of the Z line 52 to enable the pouch 110 to be formed. The stomach tissue to be folded may be drawn into the chamber 130 through the port 134 by vacuum suction and/or a mechanical tissue gripping and pulling device. Either one or both of the foregoing is contemplated in this embodiment. To that end, a vacuum may be pulled up through the elongated member 122 of the device 120. To permit this, the seal 138 may comprise a valve (not shown) to permit the vacuum to be pulled. To provide mechanical tissue gripping and pulling, the device 120, in this embodiment, further includes a helical coil 140 attached to a cable 142. The cable 142 extends from the helical coil 140 through a retractor tube 144. Helical coils for gripping tissue are well known. Hence, in this embodiment, the coil 140 may be guided out of the port 134 and into contact with the stomach tissue. Once in contact with the stomach tissue, the helical coil may be rotated to grip the tissue. Once the tissue is gripped, the retractor tube 144 and cable 142 may be displaced in a proximal direction to pull the tissue to be folded into the tissue receiving chamber 130. This may be aided by a vacuum pulled up through the elongated member 122.

Alternatively, the mechanical gripper may be used to simply pull the tissue over the port 134. From there, the vacuum may be used to pull the tissue into the chamber 130. Other combinations of the vacuum pull and mechanical grip and pull to pull the tissue to be folded into the chamber 130 are possible including, of course, employing only one such measure.

Once the tissue to be folded is within the tissue receiving chamber 130, the tissue will have facing major surfaces 150 and 152 extending substantially coextensively from a fold line 154. To maintain the tissue fold, the device 120 further includes a fastener deployer 160. The fastener deployer 160 deploys a fastener 104 through the tissue fold substantially transverse to the facing major tissue surfaces of the tissue fold 102. To this end, the fastener deployer 160 includes a guide channel 162, a fastener stylet or guide wire 166, and a fastener pusher 164. The guide wire 166 carries the fastener 102 which is pushed down the guide wire 166 by the pusher 164. The guide channel 162 may take the form of a tube, for example, to direct the stylet 166, and hence the fastener 104, across the port 134 so that the stylet 166 a fastener 104 are driven through the tissue substantially transverse to the tissue fold 102. The fastener may then be deployed as described in the copending referenced application Ser. No. 11/121,697.

FIG. 7 shows only one guide channel, fastener stylet, and fastener pusher so as to not unduly complicate the figure. It is to be understood, however, that the device 120 may include at least one other guide channel, fastener stylet, and fastener pusher. This would permit more than one fastener to be deployed for securing each stomach tissue fold as shown, for example, in FIGS. 4-6.

After the tissue fold is secured with one or more fasteners, the device is rotated an incremental amount. This causes the newly formed tissue fold to exit the port 134. It also sets the device for making another tissue fold as it incrementally moves about the circumference of the stomach.

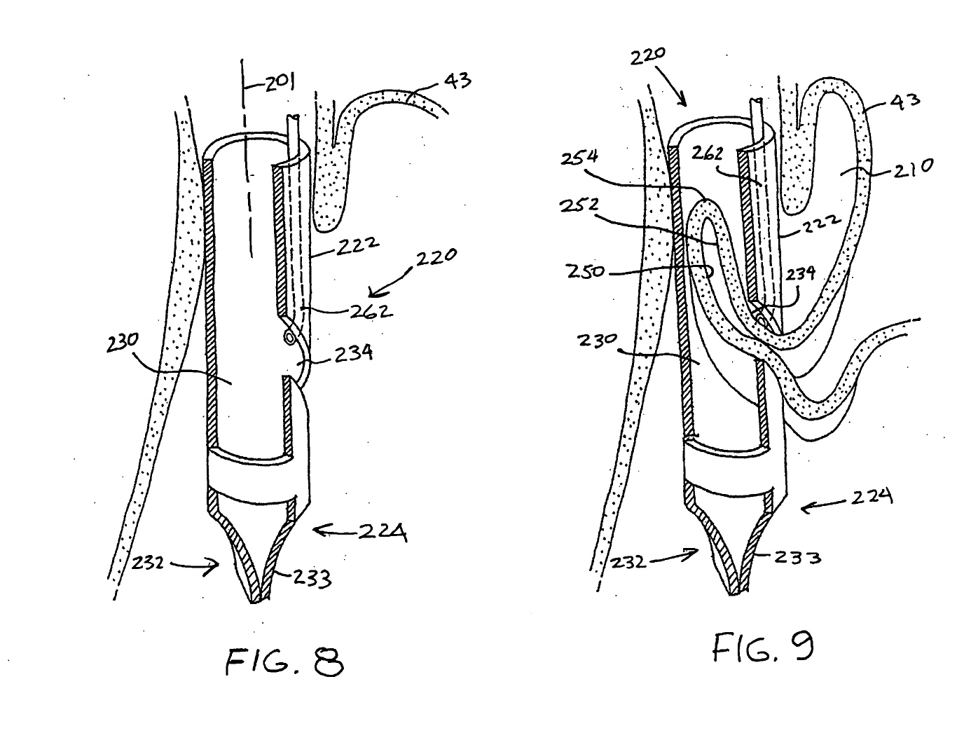

Referring now to FIGS. 8-11, they show the distal end of the elongated member 222 of a device 220 embodying the present invention. Referring first to FIG. 8, here it may be seen that the device 220 includes the elongated member 222, a tissue receiving chamber 230, a tissue receiving port 234, and a guide channel 262. The distal end has a tapered or pointed tip 232 including a duck bill valve 233. The duck bill valve 233 permits the endoscope (not shown) to extend through the elongated member 222 to enable visualization when required as previously described while providing a seal about the endoscope. When the endoscope is retracted, the duck bill valve 233 will continue to provide an effective seal. The tapered shape of the distal end of the device 220 permits the device to be guided into body spaces of limited volume.

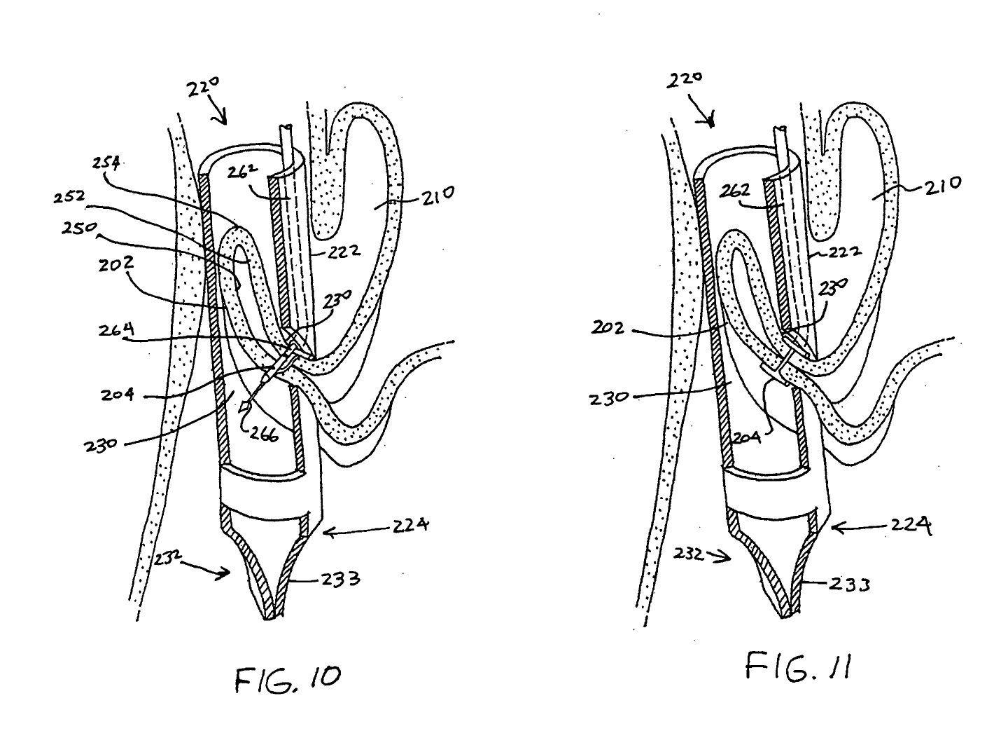

The tissue receiving port 234 is elongated for forming tissue folds as tissue is pulled into the tissue receiving chamber 230. It is also disposed at an angle to the device longitudinal axis 201. Hence, it is more suitably adapted for forming tissue folds that are non-axially arranged as shown, for example, in FIGS. 5A and 6. It may also be noted that the guide channel 262 bends as it approaches the tissue receiving port 234 to direct the fastener stylet 266 and fastener 204 substantially transversely through the tissue fold 202 as shown, for example, in FIG. 10, to be described subsequently.

As may be seen in FIG. 9, the tissue to be folded is being pulled axially up into the tissue receiving chamber 230 under a vacuum, for example. A gastric reduction pouch 210 is thus beginning to be formed. According to this embodiment, the endoscope (not shown) is first retracted from the elongated member 222 at least part way to provide additional space for the tissue to be folded. The chamber 230 is elongated having a height dimension greater than its width to accommodate folds of substantial length, if necessary. It may also be noted in FIG. 9 that the tissue to be folded is disposed within the chamber 230 by the tissue receiving port so as to have facing major surfaces 250 and 252 that extend substantially coextensively from a fold line 254.

The tissue to be folded is now ready to receive a fastener 204 to secure and complete the fold. This is illustrated in FIG. 10. Here it may be seen that the stylet 266 and fastener 204 are being directed through the tissue substantially transverse to the tissue layers. Once the fastener is deployed, the stylet 266 and pusher 264 are retracted leaving a completed fold 202 secured with a fastener 204 within the tissue receiving chamber 230. This is illustrated in FIG. 11. The tissue fold 202 will then exit the port 230 when the device 220 is rotated to begin the formation of the next tissue fold as previously described.

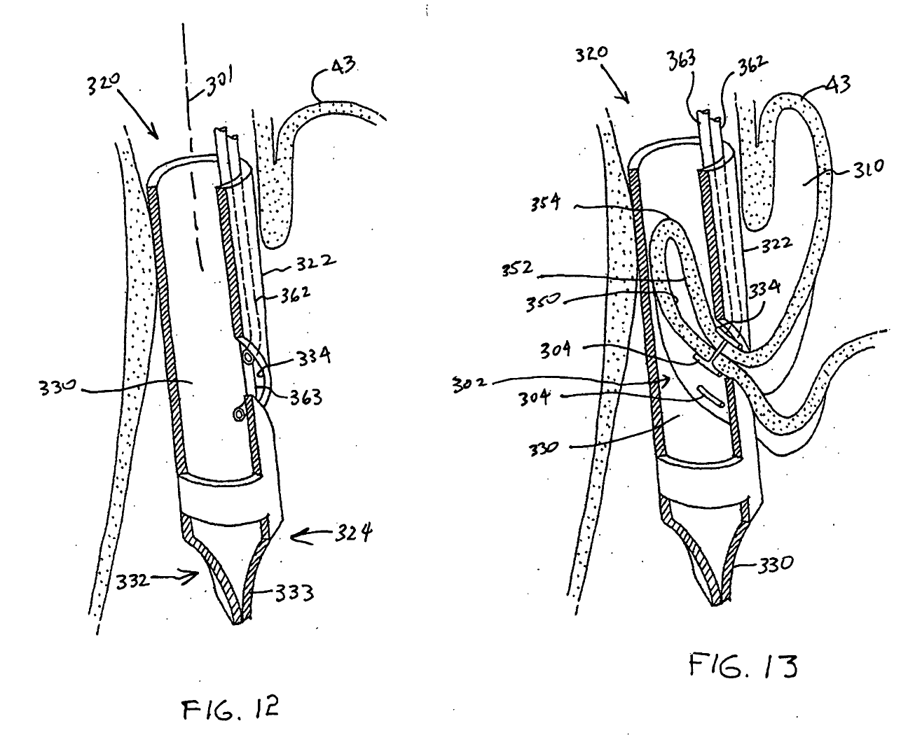

FIGS. 12 and 13 show a similar but different device 320. In FIG. 12, it may be seen that the device 320 includes the elongated member 322, a tissue receiving chamber 330, a tissue receiving port 334, and a duck bill valve 333. However, unlike the device of FIGS. 8-11, the device 320 includes a pair of guide channels 362 and 363. This permits a pair of fasteners to be deployed for securing the resulting tissue fold. The tapered distal end 332 again includes the duck bill valve 333. The duck bill valve 333 permits the endoscope (not shown) to extend through the elongated member 322 to enable visualization when required as previously described while providing a seal about the endoscope. Again, when the endoscope is retracted, the duck bill valve 333 will continue to provide an effective seal.

As in the case of device 220, the tissue receiving port 334 of the device 320 is disposed at an angle to the device longitudinal axis 301. Hence, it also is more suitably adapted for forming tissue folds that are non-axially arranged as shown, for example, in FIGS. 5A and 6. It may also be noted that the guide channels 362 and 363 bend as they approach the tissue receiving port 334 to cause the fasteners 304 to be deployed substantially transversely through the tissue fold 302 as shown, for example, in FIG. 13.

The tissue to be folded may be pulled into the tissue receiving chamber 330 and secured with fasteners 304 in the manner as described with prior reference to FIG. 10. Once the fasteners 304 are deployed, the stylets and pushers are retracted leaving a completed fold 302 secured with a pair of fasteners 304 within the tissue receiving chamber 330. The tissue is hence folded and has facing major surfaces 350 and 352 that extend substantially coextensively from a fold line 354. The tissue fold 302 will exit the port 330 when the device 320 being rotated to begin the formation of the next tissue fold as previously described. A gastric reduction pouch 310 is thus in the process of being formed.

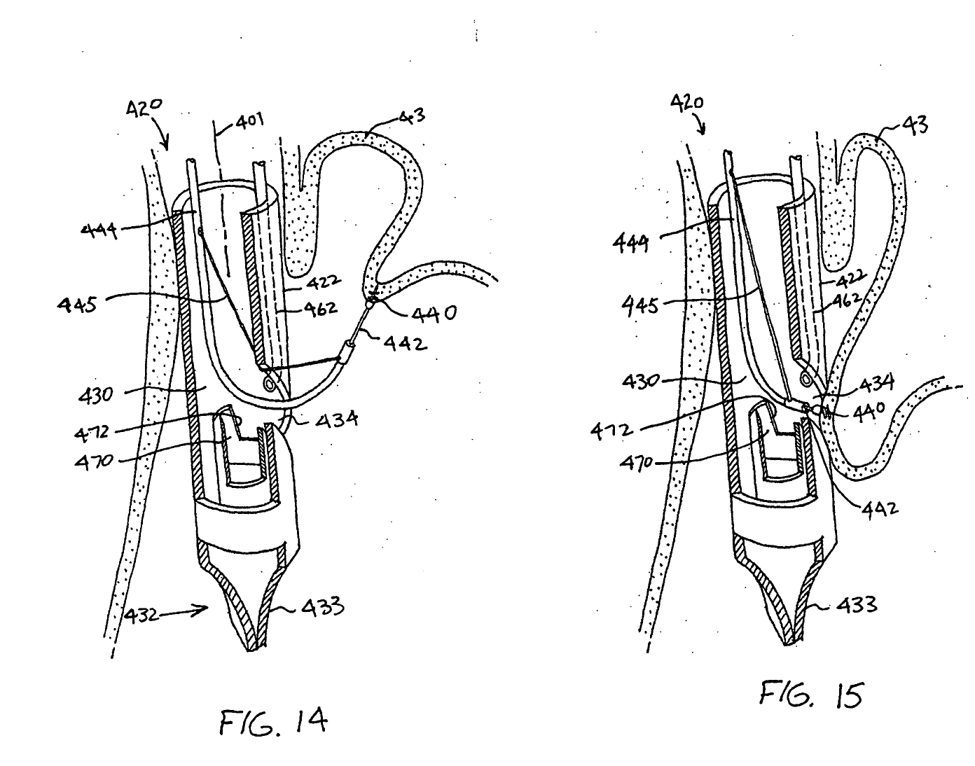

Referring now to FIGS. 14-19, they show the distal end of the elongated member 422 of still another device 420 embodying the present invention. Referring first to FIG. 14, here it may be seen that the device 420 includes the elongated member 422, a tissue receiving chamber 430, a tissue receiving port 434, and a guide channel 462. The distal end has a tapered or pointed tip 432 again including a duck bill valve 433. The duck bill valve 433 performs the same sealing functions with respect to the use and retraction of an endoscope (not shown) as previously described.

The tissue receiving port 434 is again disposed at an angle to the device longitudinal axis 401. As in previous embodiments the guide channel 462 bends as it approaches the tissue receiving port 434, again, to direct a fastener 404 substantially transversely through the tissue fold 402 as shown, for example, in FIG. 17, to be described subsequently.

Juxtaposed the delivery end of the guide channel 462 is a tissue support 470. The tissue support 470 is provided to prevent the folded tissue from tenting as the fastener is being driven through the tissue during fastener deployment. The tissue support 470 has a cut-out 472. The cut-out permits the fastener and its stylet to be driven through the folded tissue while being supported by the tissue support 470.

As may be seen in FIG. 14, the tissue to be folded is being pulled towards the tissue receiving port 434 by a mechanical puller and gripper. To provide mechanical tissue gripping and pulling, the device 420, in this embodiment, further includes a helical coil 440 attached to a cable 442. The cable 442 extends from the helical coil 440 through a retractor tube 444. A tether 445 is also provided to guide the helical coil 440 to a desired point for gripping stomach tissue.

As previously mentioned, helical coils for gripping tissue are well known. Hence, as may be contemplate by those skilled in the art, the coil 440 may be guided out of the port 434 and into contact with the stomach tissue. Once in contact with the stomach tissue, the helical coil may be rotated to grip the tissue. Once the tissue is gripped, the retractor tube 444, tether 445, and cable 442 may be displaced in a proximal direction to pull the tissue to be folded towards and to the tissue receiving port 434.

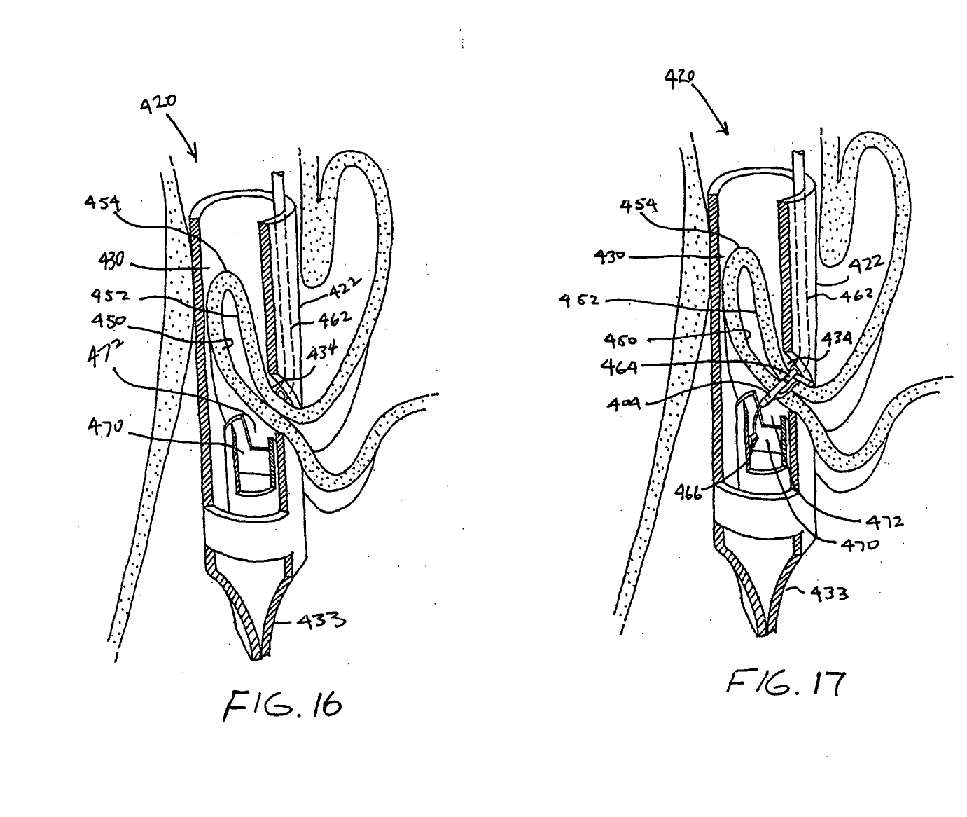

FIG. 15 shows the stomach tissue to be folded pulled up against the tissue receiving port 434 by the helical coil 440, the cable 442, the retractor 444, and the tether. The tissue is now ready to be pulled through the tissue receiving port 434 into the tissue receiving chamber 430. According to this embodiment, this final pulling is performed by pulling a vacuum in the elongated member 422. The mechanical puller may be used instead or in addition. The tissue to be folded is pulled through the tissue receiving port 434 into the tissue receiving chamber 430. As it is pulled through, it is folded by the port 434 and caused to rest against the tissue support 470. This is shown in FIG. 16.

According to this embodiment, the endoscope (not shown) is first retracted from the elongated member 422 at least part way to provide additional space for the tissue to be folded. The chamber 430, as in previous embodiments, is elongated having a height dimension greater than its width to accommodate folds of substantial length, if necessary. It may also be noted in FIG. 16 that the tissue to be folded is disposed within the chamber 430 by the tissue receiving port 434 so as to have facing major surfaces 450 and 452 that extend substantially coextensively from a fold line 454.

Referring now to FIG. 17, the tissue to be folded is now ready to receive a fastener 404 to secure and complete the fold. Here it may be seen that the stylet 466 and fastener 404 are being directed through the tissue and cut-out 472 of the tissue support 470 substantially transverse to the tissue layers and the tissue support 470. The interior of the tissue support 470 form a shield that receives the stylet 466 to protect other tissue from being pierced. Also, the support 470 bends the stylet 466 to permit the fastener delivery within the restricted space of the device 420.

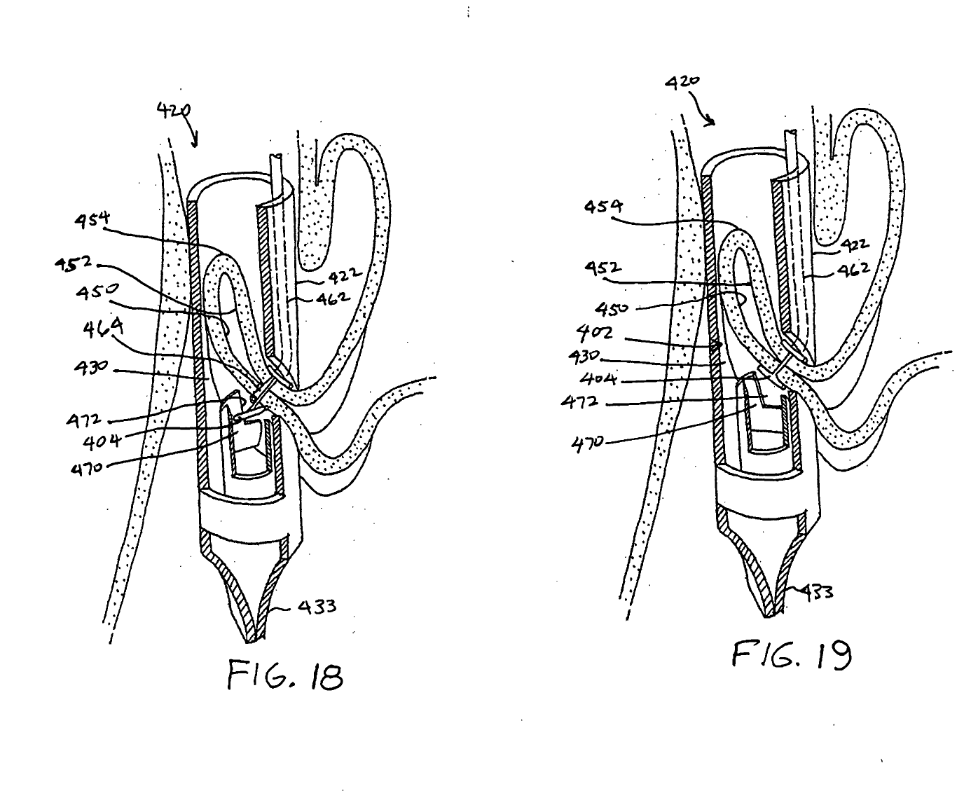

As may be seen in FIG. 18, once the stylet 466 and fastener 404 are received in the cut-out 472, the pusher 464 may push the fastener 404 off of the stylet to deploy the fastener 404. After the fastener 404 is deployed, the stylet 466 and pusher 464 are retracted leaving a completed fold 402 secured with a fastener 404 within the tissue receiving chamber 430. The tissue fold 402 will then exit the port 434. As seen in FIG. 18, the tissue support 470 is flexible at the cut-out 472, like a one-way door, for supporting the tissue as the fastener is deployed, but bending to allow the fastener 404 additional space to clear the cut-out 472 as the fold exits the port 434 as shown in FIG. 19. The device 420 may now be rotated to begin the formation of the next tissue fold as previously described.

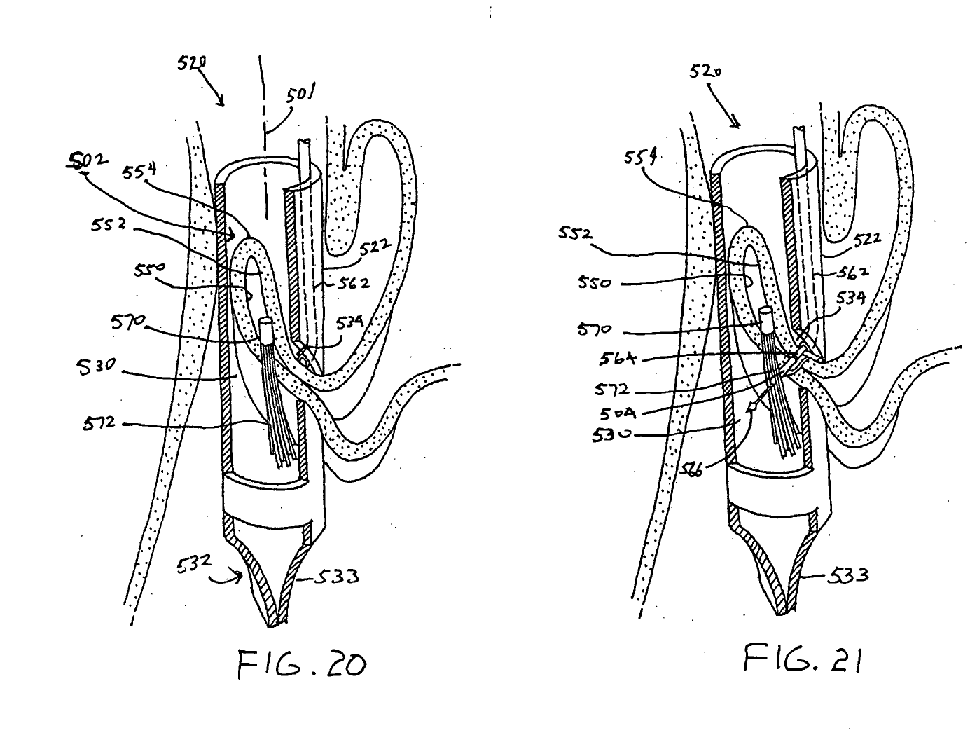

Referring now to FIGS. 20-24, they show the distal end of the elongated member 522 of still another device 520 embodying the present invention. Referring first to FIG. 20, here it may be seen that the device 520 includes the elongated member 522, a tissue receiving chamber 530, a tissue receiving port 534, a distal end seal 532, and a guide channel 562. The distal end seal 532 again takes the form of a duck bill valve 533. The duck bill valve 533 performs the same sealing functions with respect to the use and retraction of an endoscope (not shown) as previously described with respect to prior embodiments.

The tissue receiving port 534 is again disposed at an angle to the device longitudinal axis 501. As in previous embodiments the guide channel 562 bends as it approaches the tissue receiving port 534, again, to direct a fastener substantially transversely through the tissue fold 502 as shown in FIG. 20.

Juxtaposed the delivery end of the guide channel 562 is a tissue support 570. The tissue support 570 is provided to prevent the folded tissue from tenting as the fastener is being driven through the tissue during fastener deployment. The tissue support 570 has a plurality of bristles 572. The bristles permit the fastener and its stylet to be driven through the folded tissue and through the bristles 572, transverse to the bristles, while the tissue is being supported by the bristles 572 of the tissue support 570.

As previously described, the tissue to be folded may be pulled towards the tissue receiving port 534 by a mechanical puller and gripper. More specifically, the stomach tissue to be folded may be pulled up against the tissue receiving port 534 by a helical coil, a cable, a retractor, and a tether as previously described. The tissue may then be pulled through the tissue receiving port 534 into the tissue receiving chamber 530 by a vacuum and/or a mechanical puller. When the tissue to be folded is pulled through the tissue receiving port 534 into the tissue receiving chamber 530, it is folded by the port 534 and caused to rest against the tissue support 570.

As in previous embodiments, the endoscope (not shown) is first retracted from the elongated member 522 at least part way to provide additional space for the tissue to be folded. The chamber 530, as in previous embodiments, is elongated having a height dimension greater than its width to accommodate folds of substantial length, if necessary. It may also be noted in FIG. 20 that the tissue to be folded is disposed within the chamber 530 by the tissue receiving port 534 so as to have facing major surfaces 550 and 552 that extend substantially coextensively from a fold line 554.

Referring now to FIG. 21, the tissue to be folded is now ready to receive a fastener 504 to secure and complete the fold. Here it may be seen that the stylet 566 and fastener 504 are being directed through the tissue and bristles 572 of the tissue support 570 substantially transverse to the tissue layers and the tissue support bristles 572.

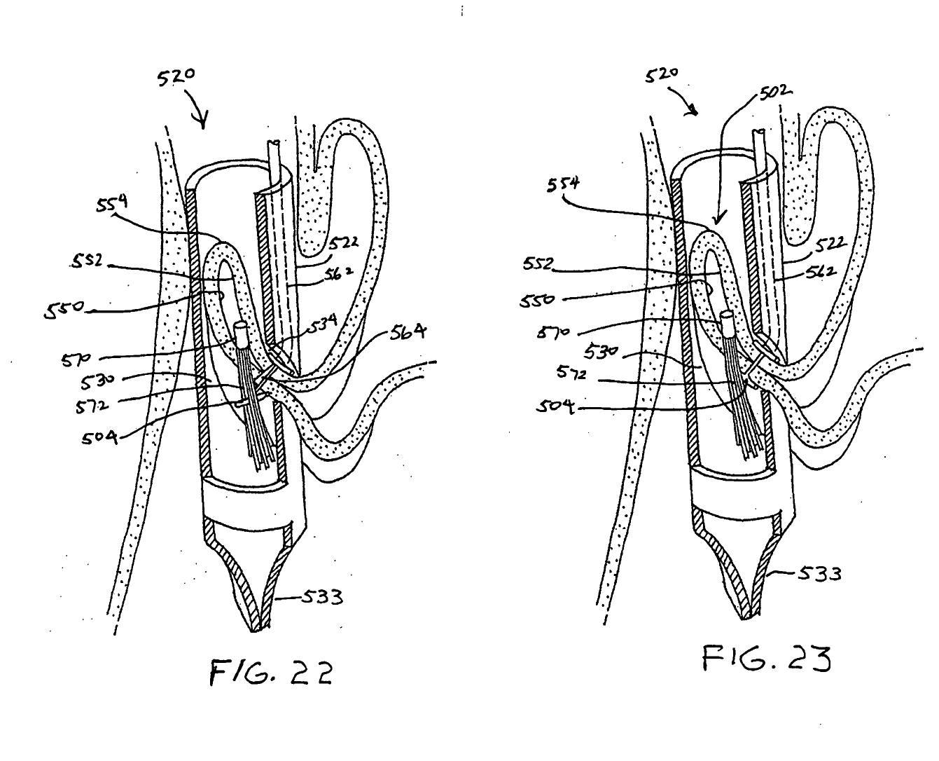

As may be seen in FIG. 22, once the stylet 566 and fastener 504 are received between adjacent ones of the bristles 572, the pusher 564 may push the fastener 504 off of the stylet to deploy the fastener 504. Although not illustrated, a support may be provided to the free end of the bristles 572 to allow the bristles to bend towards the opening when the fastener is retracted and to resist bending when the fastener is deployed. As shown in FIG. 23, after the fastener 504 is deployed, the stylet 566 and pusher 564 are retracted leaving a completed fold 502 secured with a fastener 504 within the tissue receiving chamber 530. The tissue fold 502 will then exit the port 534 when the device 520 is rotated to begin the formation of the next tissue fold as previously described.

Referring now to FIGS. 24-27, they show a fastener 604 and fastener deployment assembly 660 which may be employed according to an embodiment of the invention. The fastener 604 includes a first member 606, a second member 608, and a connecting member 610.

The first member 606 has a first end 616 and a second end 618. Similarly, the second member has a first end 612 and a second end 614. The connecting member 610 is fixed to each of the first and second members intermediate their first ends 612, 616 and second ends 614, 618.

The first member 606 includes a pointed tip 626. The pointed tip is provided to aid the fastener in piercing tissue layers to be secured. The pointed tip is preferably conical and more particularly a cone section. The pointed tip may, of course, have any one of other alternate shapes as may be appreciated by those skilled in the art.

When the fastener is fully deployed, the first member 606 and second member 608 are on opposite sides of the tissue layers with the connecting member 610 extending through the tissue there between. This may be seen in FIG. 26. Here it may be seen that the first member 606 is on one side of the tissue layers 680 and 682 and that the connecting member 610 extends between the tissue layers. To provide an increased surface area to prevent the fastener from being pulled out, the second member 608 has a plurality of segments 624. The segments are substantially in the same plane, substantially transverse from the connecting member 610, and divergent from the connecting member 610.

FIG. 25 shows an assembly 660 for deploying the fastener 606. The assembly includes a guide tube or channel 662, a tissue piercing wire or stylet 666, and a pusher 664. The fastener 604 is carried on the stylet 666 and is eventually pushed off of the stylet by the pusher 664 for deployment. To that end, the fastener 604 may be seen to include through bore or channel 622 dimensioned to slidingly receive the stylet 666. The fastener 606 further includes a slit 630 that extends continuously longitudinally along the fastener and that communicates with the through bore 622. As described in co-pending application Ser. No. 11/121,697, the slit 630 allows the fastener to slip off of the stylet 666 for deployment when pushed by the pusher 664. Reference may be had to that application for further details.

The fastener 604 is preferably a unitary structure formed of plastic. Preferably, at least the connecting member 610 and segments 624 of the second member 608 are formed of a flexible material to permit the connecting member 610 and segments 624 to bend as illustrated in FIG. 25 so as to be accommodated by the guide tube 662 along side the first member 606 and stylet 666. With this fastener configuration within the guide tube 662, the fastener is ready for deployment and my be used in any one of the embodiments previously described.

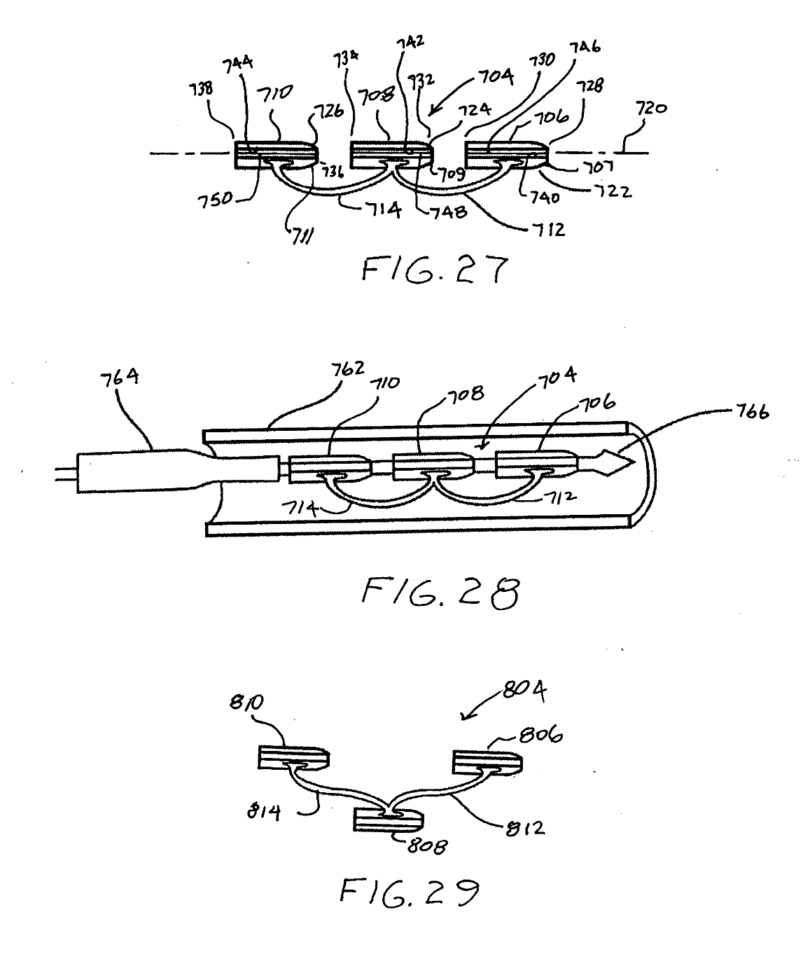

FIGS. 27 and 28 show a further fastener assembly 704 according to an embodiment of the preset invention. The fastener assembly 704 includes a first member 706, a second member 708 and a third member 710. The first member 706, second member 708 and third member 710 each have first and second ends 728 and 730, 732 and 734, and 736 and 738, respectively and are shown aligned on a common longitudinal axis 720. The assembly further includes a first connecting member 712 and a second connecting member 714. The first connecting member 712 is fixed to each of the first and second members 706 and 708 respectively intermediate their first and second ends and extends between the first and second members. The second connecting member 714 is fixed to each of the second and third members 708 and 710, respectively intermediate their first and second ends and extend between the second and third members. Hence, the first and second members 706 and 708 are separated by the first connecting member 712 and the second and third members 708 and 710 are separated by the second connecting member 714. Each of the first, second and third members 706, 708, and 710 has a through channel 740, 742, and 744, respectively, along the longitudinal axis 720 and arranged to be slidingly received on a tissue piercing deployment wire or stylet 766 (FIG. 28). Each of the first, second and third members 706, 708 and 710 has a pointed tip 707, 709 and 711, respectively, and a longitudinal slit 746, 748, and 750, respectively. The pointed tips 707, 709, and 711 are preferably formed from cone sections, are all pointed in a common direction and are provided to assist in piercing tissue to be secured. The slits 746, 748, and 750 communicate with the through bores 740, 742, and 744. This permits the assembly to be snapped onto the stylet 766 and enables each member to be pushed off of the stylet 766 during deployment.

FIG. 28 shows the fastener assembly of FIG. 27 being delivered for deployment by being pushed by a pusher 764 through a guide tube or channel 762. Here, it may be seen that all three members 706, 708, and 710 are carried in line on the stylet 766. This requires at least the connecting members 712 and 714 to be formed of a flexible material. Preferably, the assembly 704 is of unitary construction formed of plastic material. The plastic may be impregnated with a material that is at least partly radio opaque to permit the assembly 704 to be viewed under fluoroscopy.

During deployment, each member 706, 708, and 710 is pushed off of the stylet in a manner as described in the co-pending application. Here however, the member 706 is pushed by a combination of the pusher 764, the third member 710, and the second member 708. Similarly, the second member 708 is pushed by the pusher 764 and the third member 710.

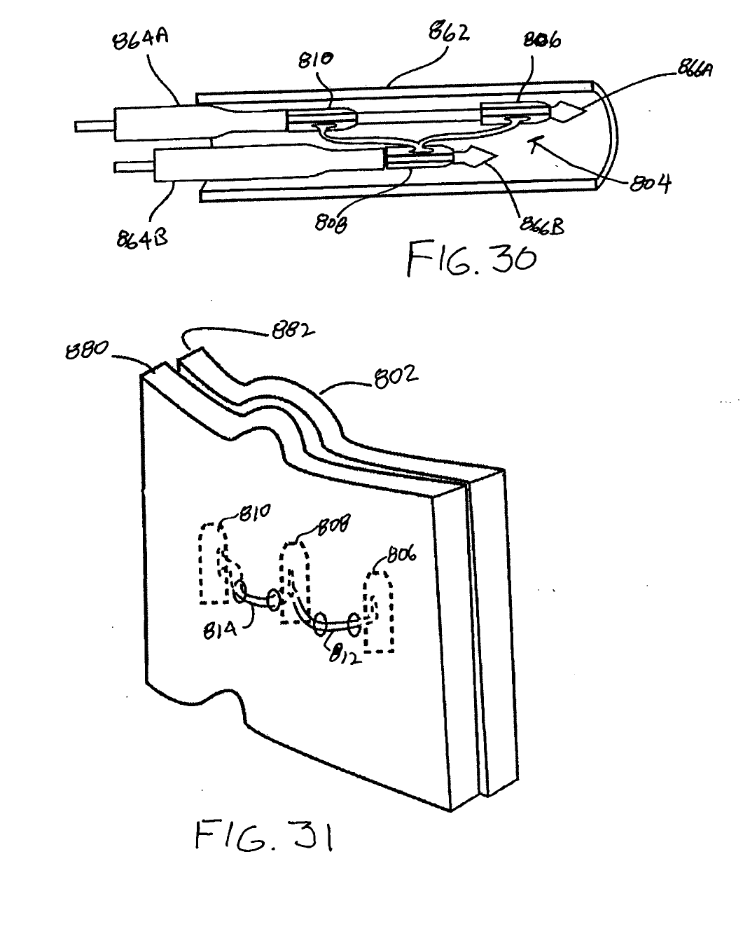

FIG. 29 shows a fastener assembly 804 that is a variation of the assembly 704 of FIG. 27. It also includes a first member 806, a second member 808, and a third member 810. However, as may be noted in FIG. 29, the first connecting member 812 and the second connecting member 814 are on the opposite side of the second member 808 as compared to the first connecting member 812 and the second connecting member 814 with respect to the second member 808. Hence, the assembly 804 is particularly adapted to be configured as shown in FIG. 30 when being delivered for deployment.

Here, it may be seen that two stylets, stylets 866A and 866B are employed. Stylet 866A carries the first member 806 and third member 810 while the stylet 866B carries the second member 808. The members 806, 808, and 810 may be pushed from their respective stylets during deployment by their respective pushers 864A and 864B.

FIG. 31 shows how the assembly 804 would appear when deployed. The assembly 704 may have a similar appearance.

Here, it may be noted that each of the members 806, 808, 810 have been driven through tissue layers 880 and 882. The connecting members 812 and 814 may be resilient to spring towards each other to create a pleat 802. The tissue layers are thus held between the members 806, 808, and 812 and the connecting members 812 and 814 as shown.

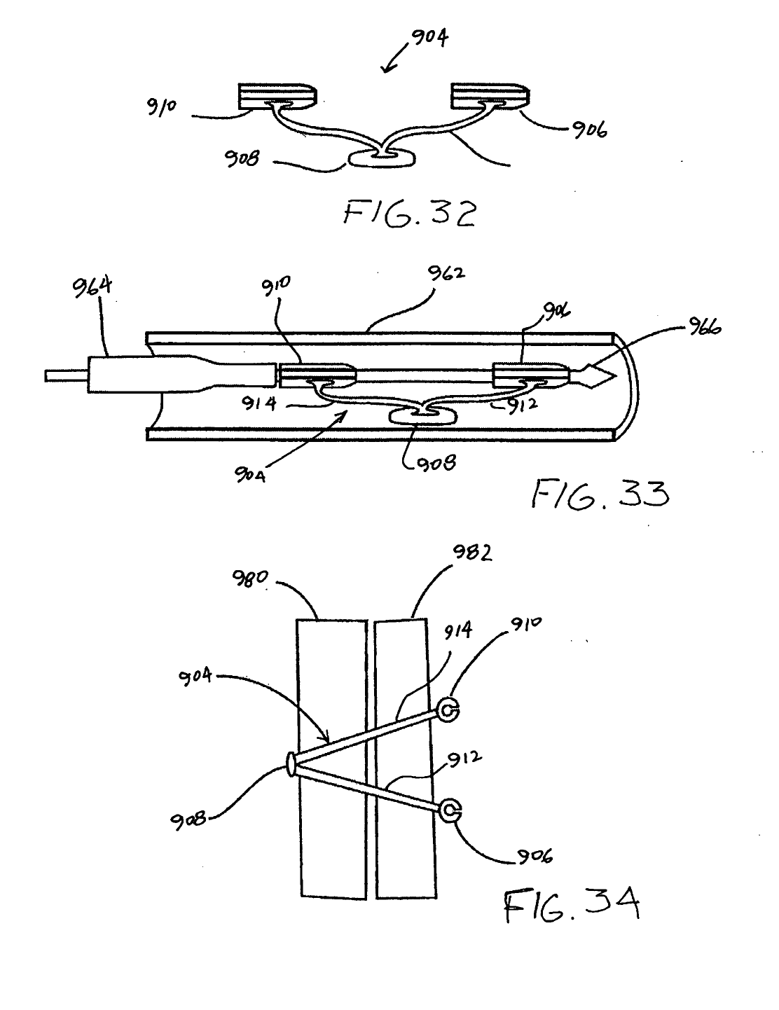

FIG. 32 shows a fastener assembly 904 that is another variation of the assembly 804 of FIG. 29. It also includes a first member 906, a second member 908, and a third member 910. However, as may be noted in FIG. 32, the second member is solid and does not include a through bore or communicating slit. Hence, like the assembly 804 of FIG. 29, the assembly 904 is particularly adapted to be configured as shown in FIG. 33 when being delivered for deployment. However, here, it may also be seen that only one stylet, stylet 966 is required for deployment. Stylet 966 carries the first member 906 and third member 910 while the second member 808 does not require, and would not accept, a stylet. The members 906 and 910 may be pushed from their stylet 966 during deployment by their pusher 964.

FIG. 34 shows how the assembly 904 would appear when deployed. Here, it may be noted the first and third members 906 and 910 have been driven through tissue layers 980 and 982. The second member 908 has not been driven through the tissue layers and thus remains on the opposite side of the tissue layers form the first and third members 906 and 910. The tissue layers 980 and 982 are thus held between the members 906 and 910 on one side and member 908 on the other side of the tissue layers 980 and 982, as shown.

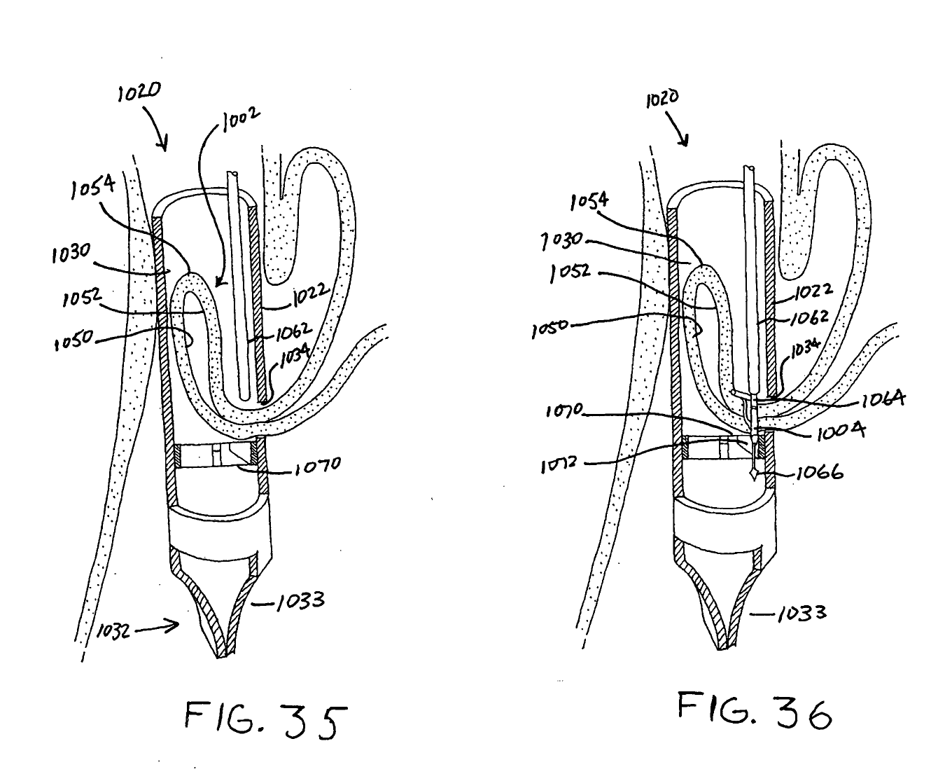

Referring now to FIGS. 35-37, they show the distal end of the elongated member 1022 of still another device 1020 embodying the present invention. Referring first to FIG. 35, here it may be seen that the device 1020 includes the elongated member 1022, a tissue receiving chamber 1030, a tissue receiving port 1034, a distal end seal 1032, and a guide channel 1062. The distal end seal 1032 is tapered and again takes the form of a duck bill valve 1033. The duck bill valve 1033 performs the same sealing functions with respect to the use and retraction of an endoscope (not shown) as previously described with respect to prior embodiments.

The tissue receiving port 1034 is disposed substantially transverse to the device longitudinal axis 1001. Since the tissue receiving port is substantially transverse to the device longitudinal axis 1001, and thus substantially horizontal, the guide channel 1062 need not bend as it approaches the tissue receiving port 1034 to direct a fastener substantially transversely through the tissue fold 1002 as shown in FIG. 36.

Juxtaposed to the delivery end of the guide channel 1062 is a tissue support 1070. The tissue support 1070 is again, as in previous embodiments, provided to prevent the folded tissue from tenting as the fastener is being driven through the tissue during fastener deployment. The tissue support 1070 has an opening 1072. The opening 1072 permits the fastener 1004 and its stylet 1066 to be driven through the folded tissue while being supported by the tissue support 1070.

As previously described, the tissue to be folded may be pulled towards the tissue receiving port 1034 by a mechanical puller and gripper. More specifically, the stomach tissue to be folded may be pulled up against the tissue receiving port 1034 by a helical coil, a cable, a retractor, and a tether as previously described. The tissue may then be pulled through the tissue receiving port 1034 into the tissue receiving chamber 1030 by a pulling and/or a mechanical puller. When the tissue to be folded is pulled through the tissue receiving port 1034 into the tissue receiving chamber 1030, it is folded by the port 1034 and caused to rest against the tissue support 1070.

As in previous embodiments, the endoscope (not shown) is first retracted from the elongated member 1022 at least part way to provide additional space for the tissue to be folded. The chamber 1030, as in previous embodiments, is elongated having a height dimension greater than its width to accommodate folds of substantial length, if necessary. It may also be noted in FIG. 36 that the tissue to be folded is disposed within the chamber 1030 by the tissue receiving port 1034 so as to have facing major surfaces 1050 and 1052 that extend substantially coextensively from a fold line 1054.

The tissue to be folded receives a fastener 1004 to secure and complete the fold. It may be seen that the stylet 1066 and fastener 1004 are being directed through the tissue and the opening 1072 of the tissue support 1070 is substantially transverse to the tissue layers and the tissue support 1070.

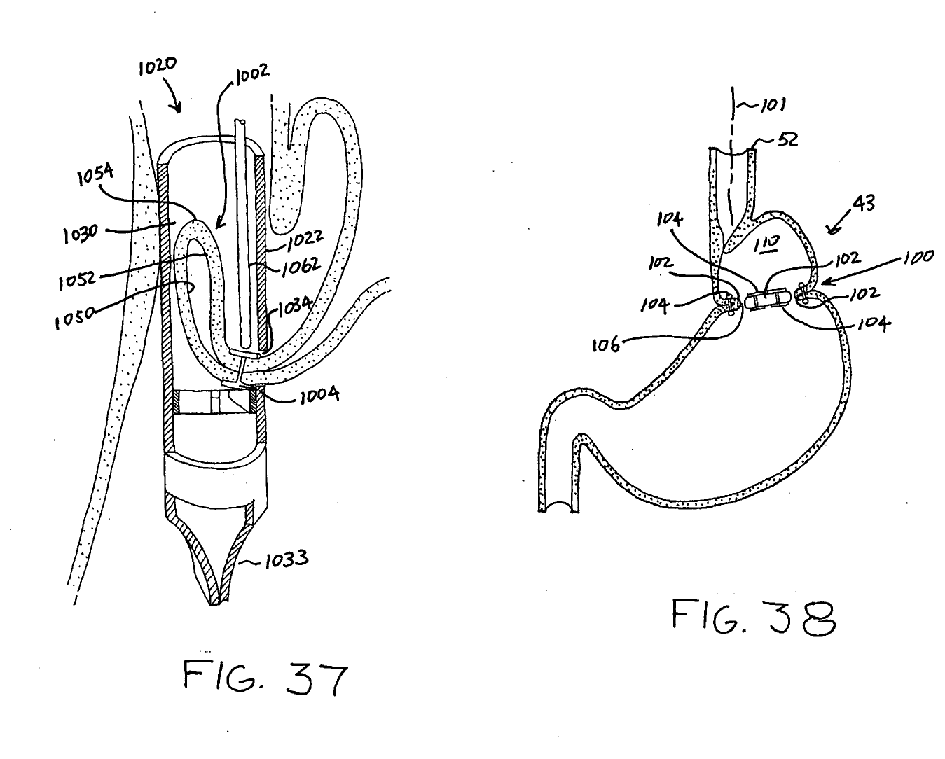

Once the stylet 1066 and fastener 1004 are received by an opening 1072, the pusher 1064 may push the fastener 1004 off of the stylet to deploy the fastener 1004. As shown in FIG. 37, when the fastener 1004 is deployed, the stylet 1066 and pusher 1064 are retracted leaving a completed fold 1002 secured with a fastener 1004 within the tissue receiving chamber 1030. The tissue fold 1002 will then exit the port 1034 when the device 1020 is rotated to begin the formation of the next tissue fold as previously described.

FIG. 38 shows a stomach with a reduced diameter portion 100 which may be formed in by the device of FIG. 35 in accordance with this embodiment of the present invention to form a gastric reduction pouch 110. As may be noted in FIG. 38, the gastric reduction pouch 110 is formed by a plurality of folds 102 made circumferentially about the stomach 43 aboral of the Z line 52. The folds 102 are formed by gathering the stomach tissue at circumferentially spaced points transorally from within the stomach.

The folds 102 are substantially in line with each other and extend longitudinally substantially transverse to the esophageal axis 101. Each stomach tissue fold may be maintained by a pair of fasteners 104 as also shown in FIG. 38. Since the folds of stomach tissue are made inwardly, the outer surfaces of the stomach tissue come into contact when the folds are fixed by the deployment of one or more fasteners 104. This presents serosa tissue to serosa tissue to aid the fasteners with the long term maintenance of the stomach tissue folds. The plurality of stomach tissue folds result in both a gastric reduction pouch 110 and an opening 106 communicating the interior of the gastric pouch with the rest of the gastric system.

While particular embodiments of the present invention have been shown and described, modifications may be made, and it is therefore intended in the appended claims to cover all such changes and modifications which fall within the true spirit and scope of the invention.

1-32. (canceled)

33. A device for forming and maintaining tissue folds from within the stomach comprising:
an elongated member having a distal end for transoral placement in the stomach;
a tissue gatherer carried on the distal end of the elongated member for placement into the stomach, the tissue gatherer defining a tissue chamber and including a tissue puller that pulls tissue into the tissue chamber to form a tissue fold within the tissue chamber; and
a fastener deployer that directs a fastener into the tissue chamber and through the folded tissue for binding the tissue fold.

34. The device of claim 33, wherein the tissue chamber is arranged to form a tissue fold having facing major tissue surfaces extending coextensively from a fold line and wherein the fastener deployer directs the fastener through the tissue substantially transverse to the facing major tissue surfaces of the tissue fold.

35. The device of claim 34, wherein the tissue gatherer is arranged for forming tissue folds that are substantially axial in orientation.

36. The device of claim 34, wherein the tissue gatherer is arranged for forming tissue folds that are substantially non-axial in orientation.

37. The device of claim 33, wherein the fastener deployer is arranged to deploy a plurality of fasteners in a tissue fold.

38. The device of claim 33, wherein the tissue chamber includes an elongated opening for receiving the stomach tissue to fold the stomach tissue.

39. The device of claim 38, wherein the elongated opening is disposed non-axially.

40. The device of claim 38, wherein the elongated member has an axis and wherein the elongated opening is disposed substantially transverse to the elongated member axis.

41. The device of claim 38, wherein the tissue puller includes a vacuum gripper and wherein the vacuum gripper pulls the stomach tissue through the elongated opening under vacuum to form the tissue folds.

42. The device if claim 41, wherein the vacuum is pulled through the elongated member and wherein the elongated member includes a valve at the elongated member distal end that seals under the vacuum.

43. The device of claim 42, wherein the elongated member has a transverse dimension configured to permit an endoscope to pass there through and through the valve when the valve is open.

44. The device of claim 38, wherein the tissue puller includes a mechanical gripper and wherein the mechanical gripper pulls the stomach tissue through the elongated opening to form the tissue folds.

45. The device of claim 44, wherein the mechanical gripper comprises a helical coil.

46. The device of claim 38, wherein the tissue chamber has a width defined by an outer wall opposite the elongated opening and length dimension, and wherein the length dimension is greater than the width dimension.

47. The device of claim 46 wherein the elongated opening has a width dimension and wherein the elongated opening width dimension and the tissue chamber width dimension are arranged to permit the stomach tissue to engage and seal against the outer wall under vacuum applied through the opening and on the stomach tissue.

48. The device if claim 33, further comprising a tissue support within the tissue chamber that supports the stomach tissue fold during fastener deployment.

49. The device of claim 33, wherein the fastener deployer includes a guide lumen that guides a fastener deployment stylet through the stomach tissue fold, the fastener deployment stylet having a distal end and arranged to guide a fastener through the stomach tissue fold.

50. The device of claim 49, wherein the tissue chamber further includes a stylet shield that receives the distal end of the fastener deployment stylet.

51. The device of claim 50, wherein the stylet shield comprises a sheath.

52. The device of claim 49, further comprising a plurality of guide lumens for guiding a like plurality of fasteners through the stomach tissue fold.

53. The device of claim 33, wherein the distal end of the elongated member includes a tapered tip.

54. The device of claim 53, wherein the tapered tip comprises a valve.

55. The device of claim 53, wherein the tapered tip comprises a duck bill valve.

56-92. (canceled)
